(19) United States
(12) Patent Application Publication
(10) Pub. No.: US 2024/0225827 A1
(43) Pub. Date: Jul. 11, 2024

(54) PERCUTANEOUSLY DELIVERABLE HEART VALVE AND METHODS ASSOCIATED THEREWITH

(71) Applicant: Colibri Heart Valve LLC, Louisville, CO (US)

(72) Inventors: R. David Fish, Houston, TX (US); David Paniagua, Houston, TX (US)

(21) Appl. No.: 18/457,575

(22) Filed: Aug. 29, 2023

Related U.S. Application Data
(63) Continuation of application No. 16/265,573, filed on Feb. 1, 2019, now abandoned.
Application No. 16/265,573 is a continuation of application No. 13/723,541, filed on Dec. 21, 2012, now abandoned.
Application No. 13/723,541 is a continuation of application No. 13/038,361, filed on Mar. 1, 2011, now Pat. No. 8,361,144.
(60) Provisional application No. 61/309,109, filed on Mar. 1, 2010.

(51) Int. Cl. A61F 2/24; A61F 2/00

(52) U.S. Cl. CPC A61F 2/2427; A61F 2/0095; A61F 2/2412; A61F 2/2415; A61F 2/2418; A61F 2/2433; A61F 2220/0075; Y10T 29/49908; Y10T 156/10

(57) ABSTRACT

An assembly configured for replacing an aortic or pulmonary heart valve in a human patient includes a transcatheter, prosthetic aortic or pulmonary heart valve. The transcatheter, prosthetic hear valve includes a frame that is expandable and collapsible, a tissue leaflet assembly sutured directly to the frame. The assembly also includes a percutaneously insertable valve delivery mechanism, wherein the transcatheter, prosthetic heart valve is releasably and coaxially mounted onto a balloon catheter of the percutaneously insertable valve delivery mechanism. The balloon catheter includes a guidewire lumen. The transcatheter, prosthetic heart valve releasably and coaxially mounted onto the balloon catheter are included in a sterile packaging to form the assembly.

### CROSS-REFERENCE TO RELATED APPLICATIONS

The present application is a continuation application of U.S. patent application Ser. No. 16/265,573, filed on Feb. 1, 2019, which is a continuation application of U.S. patent application Ser. No. 13/723,541, filed on Dec. 21, 2012, which is a continuation application of U.S. patent application Ser. No. 13/038,361, filed on Mar. 1, 2011, which claims the benefit of U.S. Provisional Patent Application No. 61/309,109 filed on Mar. 1, 2010, the contents of which are incorporated herein by reference in their entireties. Cross-reference is made to U.S. patent application Ser. No. 13/038,260 filed on Mar. 1, 2011, the content of which is incorporated herein by reference.

### FIELD

The present invention relates to the field of medical devices, and more particularly, to a percutaneously deliverable heart valve and a method of making a percutaneously deliverable heart valve.

### BACKGROUND

Heart valve disease is a common degenerative condition that compromises physiologic function and causes limiting symptoms and threat to life in millions of patients all over the world. There are various underlying causes, but malfunction of heart valves is ultimately expressed as insufficient conduction of blood through the plane of the valve due to narrowing of the anatomic pathway (stenosis), or as incompetent closure that allows blood to return back through the valve again, thereby reducing the effective forward conduction of blood through the valve (insufficiency or regurgitation). These hemodynamic states lead to 1) deficiency of cardiac output and 2) adverse loads on the pumping chambers of the heart, both of which in turn lead to functional compromise of the patient and often premature death unless effectively corrected.

Definitive corrective treatment of heart valve disease is conventionally performed by open-chest surgical techniques, wherein the valve is manipulated, repaired, or replaced with a prosthetic valve under direct vision. Heart valve surgery is performed in hundreds of thousands of cases yearly world-wide, but carries a high burden of cost, morbidity, and mortality, especially in susceptible patients who may be elderly or otherwise physiologically compromised by collateral disease. Further, the costs and resource requirements of the surgical enterprise restrict the availability of heart valve replacement to many more patients all over the world.

In pursuit of alternatives to heart valve surgery, over the last ten years a number of development programs have brought percutaneous, trans-catheter implantation of prosthetic heart valves into commercial use in the European Union (EU) and into pivotal clinical trials in the United States of America. Initial clinical experience in the EU was directed toward patients who had critical aortic valve stenosis, but were deemed to be at unacceptably high risk for open-heart surgical valve replacement. In several thousand such cases, utilizing both balloon-expandable and self-expanding designs in two separate programs, percutaneous heart valve replacement (PHVR) was shown to be feasible and possibly competitive with surgery in selected patients with 12-18 month mortality rates of about 25%. Grube E., et al., Progress and Current Status of Percutaneous Aortic Valve Replacement: Results of Three Device Generations of the CoreValve Revalving System, Circ. Cardiovasc Intervent. 2008; 1:167-175.

The application of PHVR thus far has been challenged by the technical difficulties of the implantation sequence—especially in the aortic valve position. The technique for available devices is limited by the large caliber of the devices and their delivery catheters; often, if it can be done at all in some smaller arteries, open surgical exposure and management of the femoral artery is required to insert the 18-24 French (6-8 mm diameter) systems, and their bulkiness inside the central arteries can threaten the safety of the delivery sequence. Further, access site bleeding complications form a significant part of the adverse events of the procedures.

Typically, the current PHV designs comprise a biological membrane forming the operating leaflets of the valve, attached within a metal frame, that is then collapsed onto a delivery catheter or balloon, and then constrained within an outer sheath. After an initial dilation of the diseased valve with a large balloon, this assembly is then advanced to the plane of the valve and deployed by self-expansion or by balloon expansion.

The effective caliber of the valve delivery system is determined by the total bulk of each coaxially mounted component. The bulk of the PHV itself is determined by the diameter of the frame and by the thickness, stiffness, and particular arrangement of the inner membrane forming the operating leaflets of the valve. The characteristic thickness of current PHV membranes is thus a limiting factor in the ultimate delivery profile of the PHV. Such characteristic membrane thickness is, in turn, a result of the methods by which it is processed and ultimately delivered for use. Typically, glutaraldehyde fixation (for protein cross-linking) of animal tissue is employed to produce suitable biological membranes for incorporation. Requirements for strength and durability have determined the most useful ranges for tissue thickness and cross-linking while typically imposing countervailing stiffness and brittleness. Subsequent hydration in suitable solutions improves these characteristics, but the hydrated membrane by this means also gains thickness.

One of the evident requirements for a PHV design is that the valve functions with a high degree of competence immediately on deployment, since the patient's hemodynamic survival depends on it. To this end, in part, like surgical valve prostheses, current PHV designs are completed, transported, and delivered for use in a hydrated state in a jar of solution. In use, commercially available surgical and percutaneously implanted bioprosthetic heart valves are rinsed and prepared before use in a “wet” state. More particularly, commercially available prosthetic heart valves are rinsed, crimped, and mounted in the catheterization lab. Accordingly, problems with current commercially available prosthetic heart valves include the time, cost and variability associated with the necessity to rinse, crimp, and mount the valve in the catheterization lab. That is, current mounting of prosthetic heart valves in the catheterization lab imposes one or more of delay, cost, technical burdens and possible errors. Avoiding one or more of these problems would be advantageous. In addition, current “wet” valve designs impose additional profile on the collapsed valve. The hydrated membrane, while having desirable and necessary flexibility for reliable operation immediately on deployment, also imposes a large part of the thickness of the assembled and mounted valve that compromises its deliverability.

Expanding on some of the problems described above, the use of current PHVs in the catheter lab requires a number of preparatory acts that are potentially troublesome and can prolong the delivery sequence during a critical phase of the procedure. Since PHVs are delivered for use “wet” in a preservative solution, they have to be treated prior to insertion with a series of cleansing and hydrating solutions. Once this is completed, the PHVs have to be mounted on their delivery catheters. Special crimping and mounting tools are needed in the case of the balloon-expandable Edwards Sapien valve, for example. Accordingly, there is a need to address the shortcomings discussed above.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

In at least one embodiment, an assembly configured for replacing an aortic or pulmonary heart valve in a human patient is disclosed. The assembly comprises a transcatheter, prosthetic aortic or pulmonary heart valve. The transcatheter, prosthetic aortic or pulmonary heart valve may comprise a frame that is expandable and collapsible, and a tissue leaflet assembly sutured directly to the frame. The tissue leaflet assembly may include three substantially dry leaflets formed from a treated mammalian pericardial tissue that has been treated with glutaraldehyde and glycerol, and that has a thickness of between about 50 to 300 μm. The assembly may further comprise a percutaneously insertable valve delivery mechanism including a including a balloon catheter of size 12 to 14 French, and that includes a guidewire lumen. The transcatheter, prosthetic aortic or pulmonary heart valve may be releasably and coaxially mounted onto the balloon catheter of the percutaneously insertable valve delivery mechanism. The assembly may also include a sterile package containing the transcatheter, prosthetic aortic or pulmonary heart valve that is releasably and coaxially mounted onto the balloon catheter.

In certain embodiments, the frame may comprise a stent.

In some embodiments, the treated mammalian pericardial tissue may have an ultimate tensile strength of greater than about 12 MegaPascals.

In at least one embodiment, the treated mammalian pericardial tissue may not include a matrix that has been exposed to a polymer infiltrate.

In one or more embodiments, the treated mammalian pericardial tissue may have been treated with distilled water. Additionally and/or alternatively, the treated mammalian pericardial tissue may have been treated with isopropyl alcohol.

### BRIEF DESCRIPTION OF THE DRAWINGS

To further clarify the above and other advantages and features of the one or more present inventions, a more particular description of the one or more present inventions is rendered by reference to specific embodiments thereof which are illustrated in the appended drawings. It is appreciated that these drawings depict only typical embodiments of the one or more present inventions and are therefore not to be considered limiting of its scope. The one or more present inventions is described and explained with additional specificity and detail through the use of the accompanying drawings in which:

FIG. 1 is a flow chart of a method associated with at least of one embodiment of the present invention;

FIGS. 2A-2B are a flow chart illustrating elements of the tissue preparation;

FIG. 3 is a flow chart illustrating elements of the drying and sizing;

FIG. 4 is a flow chart illustrating elements of the valve construction with attachment of tissue membrane leaflets to a frame;

FIG. 5 is a flow chart illustrating elements of the mounting of the valve into a delivery system;

FIG. 6 is a flow chart illustrating elements of the ensheathing, sterilization, and packaging;

FIG. 7 is a flow chart illustrating elements of the delivery of the valve into a patient;

FIG. 8A is a view of a one-piece section of tissue prior to being folded;

FIG. 8B is a view of two (of three) separate pieces of tissue after folding (detailed below);

FIG. 8C is a view of the two pieces of tissue shown in FIG. 8B after being sutured together at the pleat formed after folding (detailed below);

FIG. 8D is a view of a tissue blank with the line of primary fold shown using a dashed line;

FIG. 8E is a perspective view of the tissue blank being folded along the primary fold line;

FIG. 8F is a 2-part figure showing the pleats fold lines and pleats after folding;

FIG. 8G is a detail perspective view of a single pleat shown in FIG. 8F;

FIG. 8H is a perspective schematic view of a folded and seamed tissue leaflet assembly;

FIG. 8I is a perspective schematic view of a frame;

FIG. 8J is a perspective schematic view of the frame of FIG. 8I with the tissue leaflet assembly of FIG. 8H attached thereto;

FIG. 8K is side elevation schematic view of the device shown in FIG. 8J;

FIG. 8L is an end schematic view of the frame and tissue leaflet assembly attached thereto;

FIG. 9 is a graph that shows actual stress-strain test results for five tissue samples prepared in accordance with at least one embodiment;

FIG. 10 is a schematic of a portion of a catheter with a percutaneously deliverable heart valve mounted thereto;

FIG. 11A is a photo of an implantable prosthetic heart valve, including a tissue leaflet assembly attached within a frame, wherein the tissue is situated in a partially open orientation;

FIG. 11B is a drawing of an implantable prosthetic heart valve, including a tissue leaflet assembly attached within a frame, wherein the tissue is situated in a closed orientation;

FIG. 11C is a side cutaway view of an implantable prosthetic heart valve, including a tissue leaflet assembly attached within a frame, wherein the tissue is situated in a closed orientation;

FIG. 11D is another side cutaway view of an implantable prosthetic heart valve, including a tissue leaflet assembly attached within a frame, wherein the tissue is situated in a closed orientation;

FIG. 12 is a photo of valve tissue after testing through 30,000,000 cycles of pumping used to model human heart conditions, wherein the photo shows a smooth uniform surface;

FIG. 13 is a drawing of a surgeon holding a premounted percutaneously deliverable heart valve associated with a catheter and residing within sterile packaging;

FIG. 14 is a schematic of a simplified cutaway view of a human heart, including heart valves that may be targeted for receiving an embodiment of an implantable prosthetic heart valve;

FIG. 15 is a schematic of a human aorta receiving a catheter with an implantable prosthetic heart valve mounted thereto;

FIG. 16 is a schematic of a human aorta with the implanted prosthetic heart valve implanted at the site of the original diseased aortic valve; and

FIG. 17 is a schematic of a prosthetic heart valve and a mandrel of a delivery catheter.

The drawings are not necessarily to scale.

### DETAILED DESCRIPTION

Embodiments of the one or more inventions described herein include one or more devices, assemblies and/or methods related to a prosthetic heart valve. A prosthetic heart valve in accordance with at least one embodiment described herein can be surgically implanted, such as by percutaneous, trans-catheter delivery, to the implantation site within the patient. One or more embodiments of the prosthetic heart valves described herein have application for at least aortic and pulmonary valve positions, including for structural defects and diseased valves.

In at least one embodiment, biocompatible material is attached within a frame to form an implantable prosthetic heart valve, and then at a later time, the implantable prosthetic heart valve is implanted within a patient, such as by way of a percutaneous, trans-catheter delivery mechanism. Once implanted, the prosthetic heart valve serves to regulate the flow of blood associated with the patient's heart by allowing forward blood flow and substantially preventing backflow or valvular regurgitation.

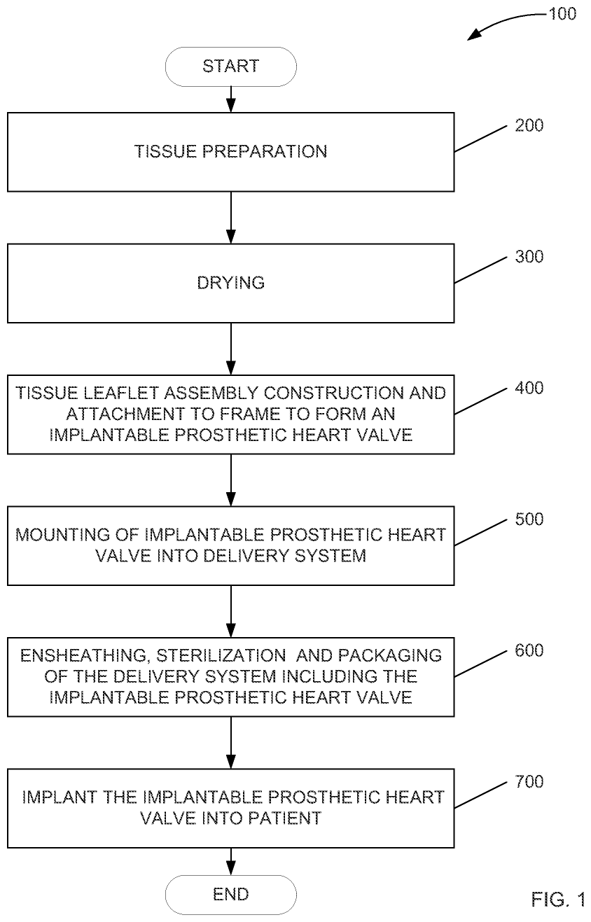

Referring now to FIG. 1, a flow chart illustrates at least one embodiment of a prosthetic heart valve preparation and delivery method 100. The prosthetic heart valve preparation and delivery method 100 generally includes a plurality of procedures to include tissue preparation at 200, drying at 300, tissue leaflet assembly construction and attachment to frame at 400 to form an implantable prosthetic heart valve, mounting of the prosthetic heart valve (that is, the frame with the tissue leaflet assembly) into a delivery system at 500, ensheathing, sterilizing and packaging the delivery system including the prosthetic heart valve at 600, and finally, delivering the prosthetic heart valve into the patient at 700. Further detail of the prosthetic heart valve preparation and delivery method 100 is provided below.

At least one or more embodiments described herein include a relatively thin tissue component. By way of example and not limitation, in at least one embodiment the tissue has a thickness of approximately 50-150 μm, and further possesses characteristics of pliability and resistance to calcification after implantation. The relatively thin nature of the tissue used in the implantable prosthetic heart valve assists with biocompatibility. In addition, the relatively thin tissue component thereby provides for a relatively low mass. As a result, an implantable prosthetic heart valve using the tissue can accelerate to a relatively high heart rate in beats per minute with competent function.

Tissue suitable for use in the one or more prosthetic heart valves and/or one or more assemblies described herein is relatively thin and can generally be considered to be a membrane. Those skilled in the art will appreciate that both natural and synthetic types of materials may be used to form a leaflet assembly of a prosthetic heart valves. Accordingly, it is to be understood that although treated pericardium tissue is described as a suitable material for use in the leaflet assembly of a prosthetic heart valve of one or more embodiments described herein, material other than xenograft tissue membrane can be used, and indeed, xenograft tissue membrane other than pericardium tissue can be used. More specifically, synthetic materials may include, but are not limited to, PTFE, PET, Dacron, and nylon. In addition, other than pericardium tissue, xenograft tissue membrane may include, but is not limited to, membrane material from the intestine, lung and brain. Suitable material may also comprise allograft material, that is, material from human sources. The listing of possible materials is for exemplary purposes and shall not be considered limiting.

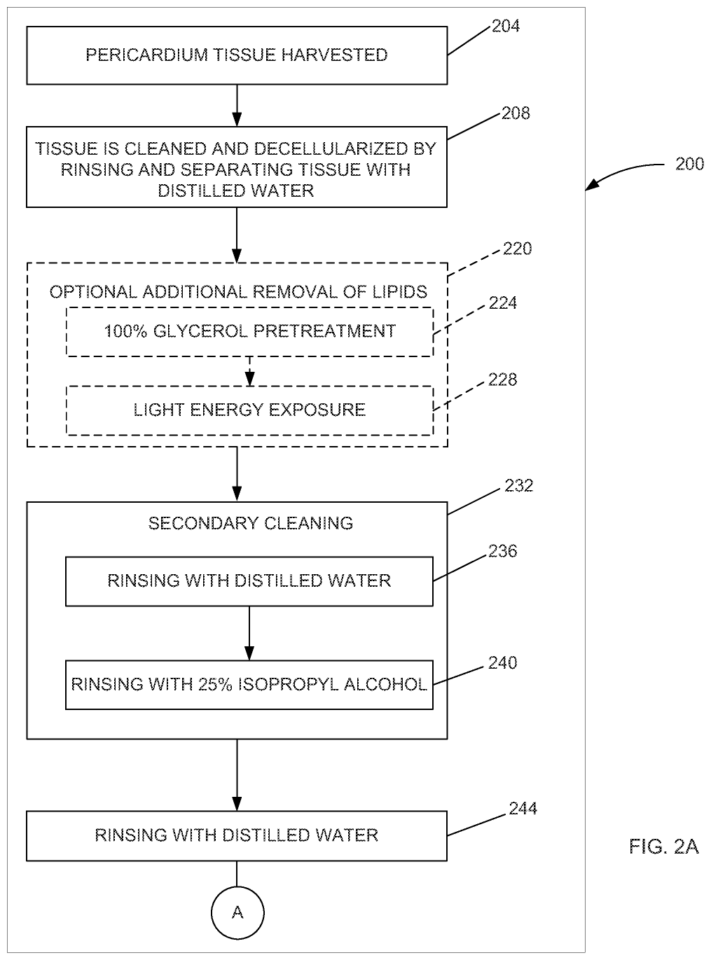

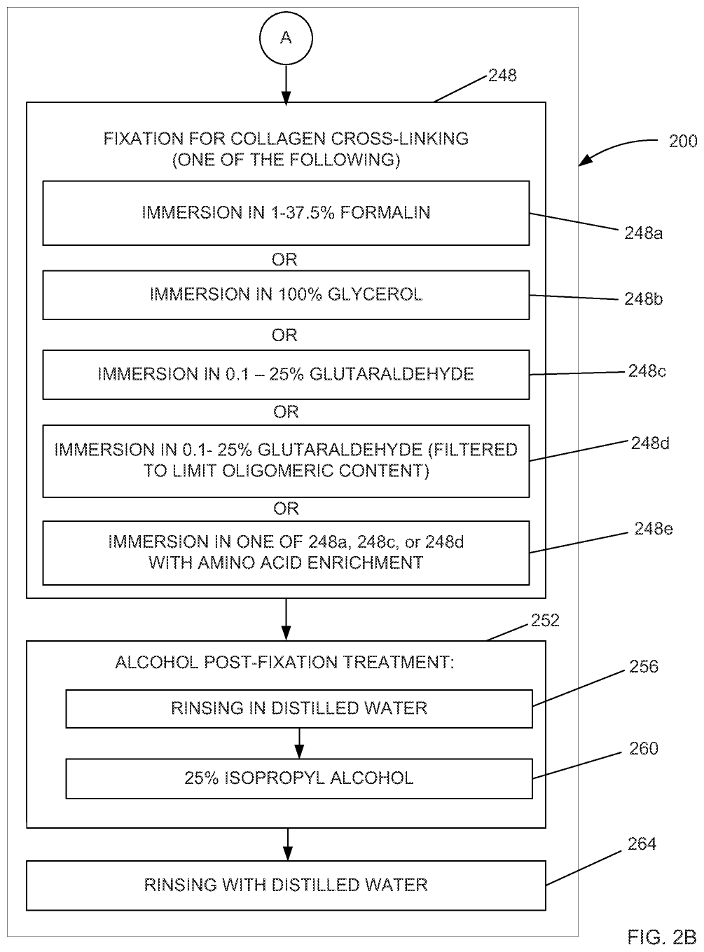

With reference now to FIG. 2A, the process associated with preparation of a biocompatible tissue consistent with the above-noted characteristics is described. In at least one embodiment, pericardium tissue, such as porcine or bovine pericardium tissue, is harvested at 204 and then processed to serve as the biocompatible tissue for association with a frame, such as by attaching within a frame. Accordingly, subsequent to the harvesting at 204, the pericardium tissue is cleaned and decellularized at 208. More particularly, in at least one embodiment the tissue is initially cleaned with distilled water using gentle rubbing and hydrodynamic pressure at 208 in order to remove adherent non-pericardial and non-collagenous tissue. In at least one embodiment, the hydrodynamic pressure at 208 is provided by spraying the tissue with a relatively weak stream of liquid to remove at least some of the non-collagenous material associated with the tissue. The rinsing at 208 is to achieve effective decellularization of the pericardium tissue through osmotic shock. Typically, the thickness of the tissue in the cleaned condition varies from about 50 to 500 micrometers, depending on the source of raw tissue. Cleaning preferably continues until there is no visible adherent non-pericardial or non-collagenous tissue.

With continued reference to FIG. 2A, after the tissue has been cleaned and decellularized at 208, the tissue then undergoes optional additional removal of lipids at 220 to further treat the tissue for preventing immunologic response and calcification. More particularly, the tissue first optionally undergoes a 100% glycerol pretreatment at 224 while being positioned on a flat surface (e.g., an acrylic plate), after which the tissue becomes nearly transparent.

At 228, the tissue optionally undergoes a “thermophotonic” process. In at least one embodiment, the tissue is optionally exposed to light energy for additional removal of lipids and for initial cross-linking of the collagen. By way of example and not limitation, in at least one embodiment a 25-100 watt incandescent light source, and more preferably, a 50 watt incandescent light source with a flat radiant face is employed at a distance of about 10 centimeters from the tissue surface, typically requiring 15 minutes of exposure before further visible separation of lipid droplets from the tissue stops.

Still referring to FIG. 2A, the tissue is then cleaned again in secondary cleaning at 232. More particularly, at 236 the tissue is again rinsed with distilled water. Thereafter, at 240 the tissue is rinsed with 25% isopropyl alcohol for periods of several hours to several days and weeks, depending on the desired tissue properties of pliability and tensile strength. By way of example and not limitation, tissue has been successfully prepared by rinsing with 25% isopropyl alcohol for a period of 7 days, and after further treatment steps described herein, provided an ultimate tensile strength of greater than 25 MegaPascals. Here, the combination of tissue pliability and tensile strength is sought for purposes of producing a material having property characteristics suitable for being physically manipulated to form a tissue leaflet assembly or other configuration appropriate for attaching with a frame, while providing a tissue material that will operate properly once implanted. These techniques are intended to conserve and preserve collagen fibers, minimizing damage to the tissue and improving tissue characteristics. The preparation and fixation techniques produce tissue membrane material that may be rendered and used at lesser thickness than typically rendered in the prior art. Thinner membranes are more pliable, but with conventional preparation techniques the tensile strength of the tissue is sacrificed. Advantageously, the preparation techniques described herein have produced membranes that have as much as three times the tensile strength of a commercial product of the prior art. This achieved strength is thus enabling for providing a tissue leaflet assembly having a low profile with appropriate durability, even in a substantially dry state. More particularly, the tissue possesses a relatively high tensile strength. By way of example and not limitation, testing has shown that embodiments of tissue prepared as described herein provide a tissue with a tensile strength of approximately three times the tensile strength of current pericardial valve tissue, such as on the order of approximately 25 MegaPascals, thereby providing about 2000 times the physiologic load strength for valve tissue. Moreover, testing of an embodiment of an implantable prosthetic heart valve made with tissue prepared as described herein and under a static load of greater than approximately 250 mmHg showed less than approximately 14% leakage, wherein such results are generally considered superior to surgical tissue valve prostheses.

In at least one embodiment where isopropyl alcohol is described as a rinsing agent, ethanol may be used in its place as an alternative, although resulting tissue properties may vary.

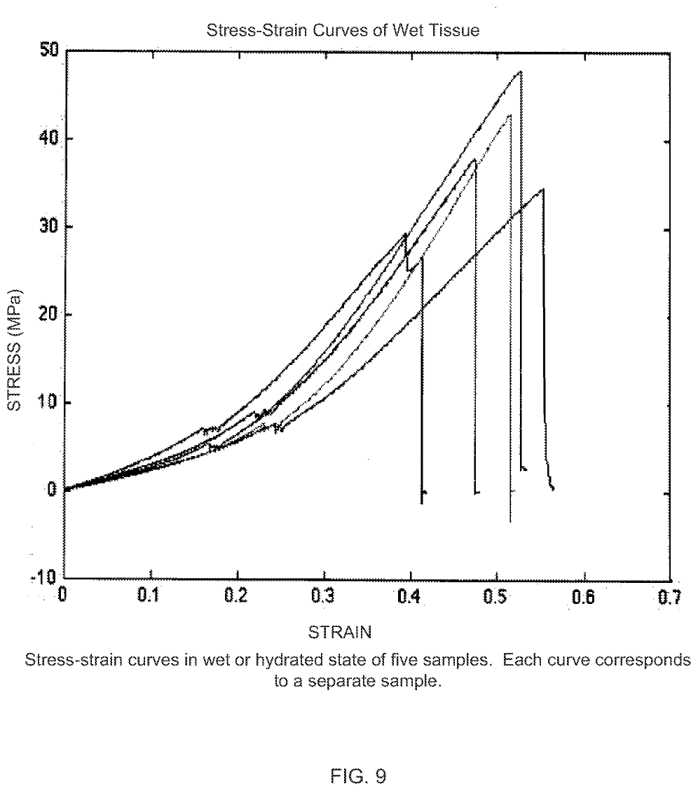

With reference to FIG. 9, stress-strain curve results for five different tissue samples prepared in accordance with an embodiment are shown. For the testing results shown, the yield stress or ultimate tensile strength was obtained by mounting strips of tissue fixed at the ends in a linear force tester and increasing the length by 0.3 mm/sec while recording resultant force (tension) until the material ruptured or separated entirely; these measurements were then used to calculate the stress-strain curves depicted in FIG. 9. As illustrated in the graph, the yield stress or ultimate tensile strength of the various tissue samples varied from about 30 to about 50 MegaPascals. More particularly, for each curve shown in FIG. 9, the testing procedures were the same. That is, each of the curves shown pertain to separate pieces of tissue that were subjected to the same test. The results show a minimum ultimate tensile strength of 30 MegaPascals, with a range up to 50 MegaPascals. Accordingly, the illustrated test results demonstrate consistency of the ultimate tensile strength results for the tissue treatment process.

With reference back to FIG. 2A, the tissue is rinsed with distilled water at 244 as a final cleaning step and for rehydration.

Referring now to FIG. 2B, following the rinse with distilled water at 244, treatment of the tissue continues. More particularly, fixation for collagen cross-linking at 248 is achieved by performing at least one of the following:
a. At 248a, immersion of the tissue in 1-37.5% formalin, ideally a buffered solution, for between about 3 days to 5 weeks, and more preferably, for between about 3 days to 4 weeks, and more preferably yet, for between about 3 weeks to 4 weeks, at a temperature of between about 4 to 37° C., and more preferably, 10% formalin for 6 days at 20° C.; or
b. At 248b, immersion of the tissue in 100% glycerol for up to 6 weeks at between 4 to 37° C., and more preferably, immersion of the tissue in 100% glycerol for about 3 weeks at 20° C.; or
c. At 248c, immersion of the tissue in 0.1-25% glutaraldehyde for between about 3 days to 5 weeks, and more preferably, for between about 3 days to 4 weeks, and more preferably yet, for between about 3 weeks to 4 weeks, at 0 to 37° C., and more preferably, immersion of the tissue in 0.25% glutaraldehyde for 7 days at 4° C.; or
d. At 248d, immersion of the tissue in 0.1-25% glutaraldehyde (filtered to limit oligomeric content) for between about 3 days to 5 weeks, and more preferably, for between about 3 days to 4 weeks, and more preferably yet, for between about 3 weeks to 4 weeks, at 0 to 37° C., and more preferably, 0.25% glutaraldehyde for 7 days at 4° C.; or
e. At 248e, immersion in the tissue in one of the above formalin, glutaraldehyde, or oligomeric filtered glutaraldehyde solutions together with added amino acids, lysine and/or histidine, wherein the concentration of the amino acids, L-lysine or histidine, used as an additive to the fixative is in the range of about 100-1000 milliMolar, with a preferred value of about 684 mM.

In addition to the foregoing, combinations of the processes listed above may be performed, including: step a followed by step b; step a followed by step c; and step a followed by step d.

As those skilled in the art will appreciate, heat-shrink testing may be conducted on tissue samples to correlate the effectiveness of protein cross-linking. Here, results of heat-shrink testing performed on one or more samples of tissue prepared in accordance with at least one embodiment using formalin showed that the tissue had a shrink temperature of 90° C. This compares favorably with samples prepared using glutaraldehyde, wherein the shrink temperature was 80° C. Accordingly, formalin is a suitable variant of fixation. It is noted that formalin was generally abandoned by the field, largely because of material properties that were unfavorable and because of inadequate or unstable protein cross-linking. Such problems have been overcome through the pretreatments described herein, allowing production of tissue with strength, pliability, and durability in a relatively thin membrane. When used in a percutaneous deliverable heart valve (also referred to herein as “prosthetic heart valve”), the tissue characteristics imparted by the tissue preparation process facilitate formation of a construct having a relatively low-profile, which also thereby facilitates dry packaging of the prosthetic heart valve. The same advantages are also achieved using the pretreatments when using a glutaraldehyde process.

Referring still to FIG. 2B, after fixation for collagen cross-linking at 248, an alcohol post-fixation treatment at 252 is preferably performed by rinsing the tissue in distilled water at 256, and then at 260 rinsing the tissue in 25% isopropyl alcohol for between about 30 minutes to 14 days or more at between about 0 to 37° C., and more preferably, for at least about 7 days at 20° C. At 264, the tissue undergoes a rinsing with distilled water.

In accordance with at least one embodiment, treatment of the tissue, including from the time of harvest to the time of implantation or grafting, does not include contact and/or exposure to a polymer to infiltrate and/or encapsulate tissue fibers of the tissue.

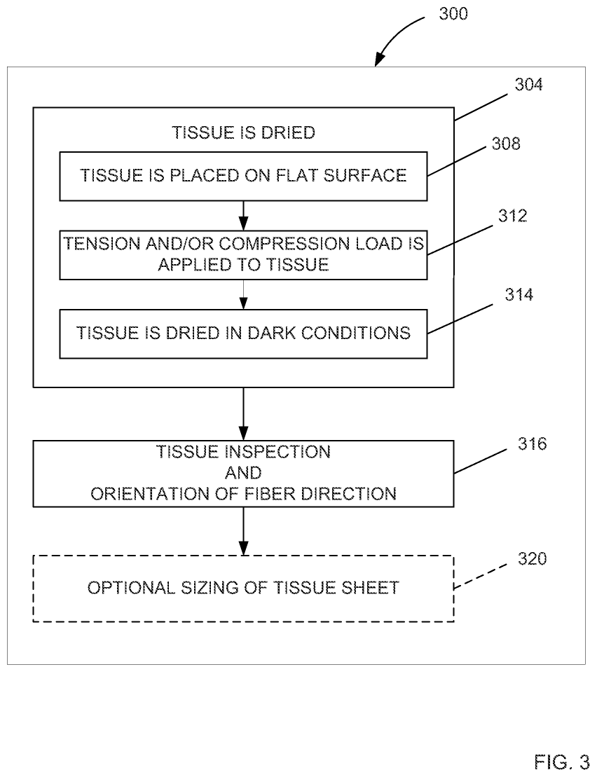

Referring now to FIGS. 1 and 3, the drying process at 300 is performed after the tissue preparation at 200. Thus, in accordance with at least one embodiment, the tissue is dried under a load. More particularly, for the tissue drying at 304, the tissue is placed minimally stretched flat (that is, stretched just enough to eliminate visible wrinkles and bubbles) on a flat surface (e.g., a polymer or acrylic sheet) at 308, and held fixed at its edges at 312. Optionally, the joined tissue and underlying sheet are then set in a slight curve. The tension maintains the substantially flat structure of the tissue as it dries, thereby mitigating or preventing excessive shrinkage, wrinkling, and/or curling at the edges, and also making the rate of drying more uniform across the surface of the tissue because of the surface tension between the plate and the tissue. Alternatively, the tissue is dried while compressed between acrylic plates. When drying the tissue, the temperature is held at between about 4 to 37° C., and more preferably, between about 20 to 37° C. (i.e., approximately room temperature to normal human body temperature), and more preferably, at about 20° C. At 314, the drying process is performed in substantially dark conditions (i.e., substantially no visible light) for between about 6 hours to 5 days, and more preferably, for about 72 hours. By way of example, the tissue is dried in dark conditions at a temperature of about 20° C. for between about 6 hours to 5 days, and more preferably, for about 72 hours. As those skilled in the art will appreciate, drying the tissue while the tissue is compressed between plates requires a longer period of time.

In at least one embodiment, after drying, the tissue lots are inspected at 316, such as by stereomicroscopy, to identify and discard those with defects or discontinuities of the fiber matrix. In addition, the preferential fiber direction for each piece is identified to determine the necessary orientation of the free edge of the pieces that will form the valve leaflets. Depending upon the size (i.e., the area) of the tissue being prepared and the size of tissue needed for a given valve, the tissue may be trimmed or otherwise sized in optional sizing at 320, such as by cutting the tissue into an appropriately sized and shaped sheet for valve formation. Preferably, cutting of the tissue membrane is oriented so that the resulting free edge of the leaflet is parallel to the preferential fiber direction of the tissue membrane. Optionally, the free edge of the leaflets may also be cut with a parabolic or other curved profile to compensate for the downward angle from the commissural leaflet attachment point to the central coaptation point and to increase the total contact surface between the coapting leaflets. This approach minimizes focal weaknesses in the operating margins of the leaflet assembly and advantageously distributes the principal loading forces of the operating valve along the long axis of the collagen fibers. As a result, the tissue is resistant to surface fracture and fraying. As shown in FIG. 3, optional sizing at 320 is performed after the drying at 304 and inspection at 316.

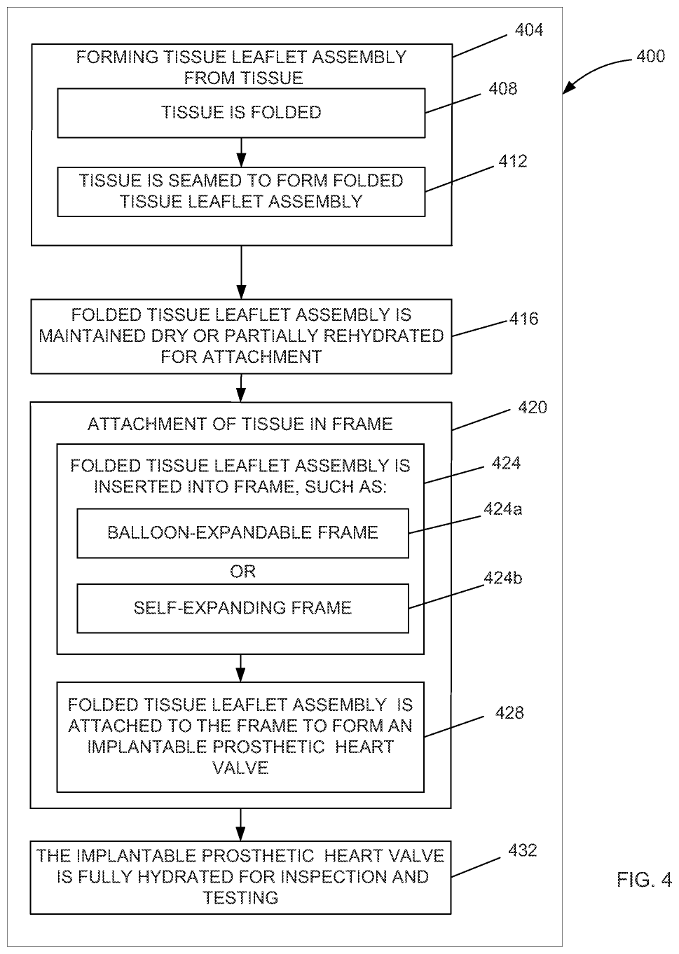

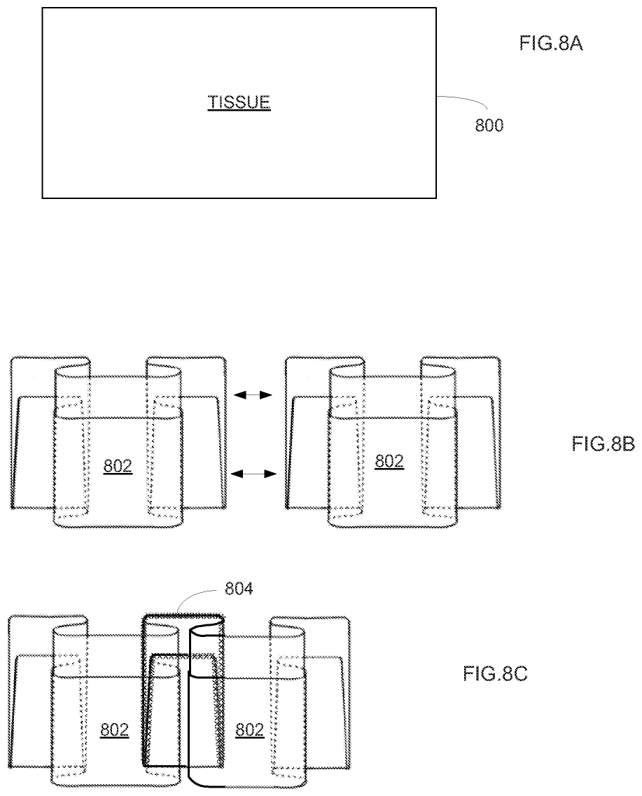

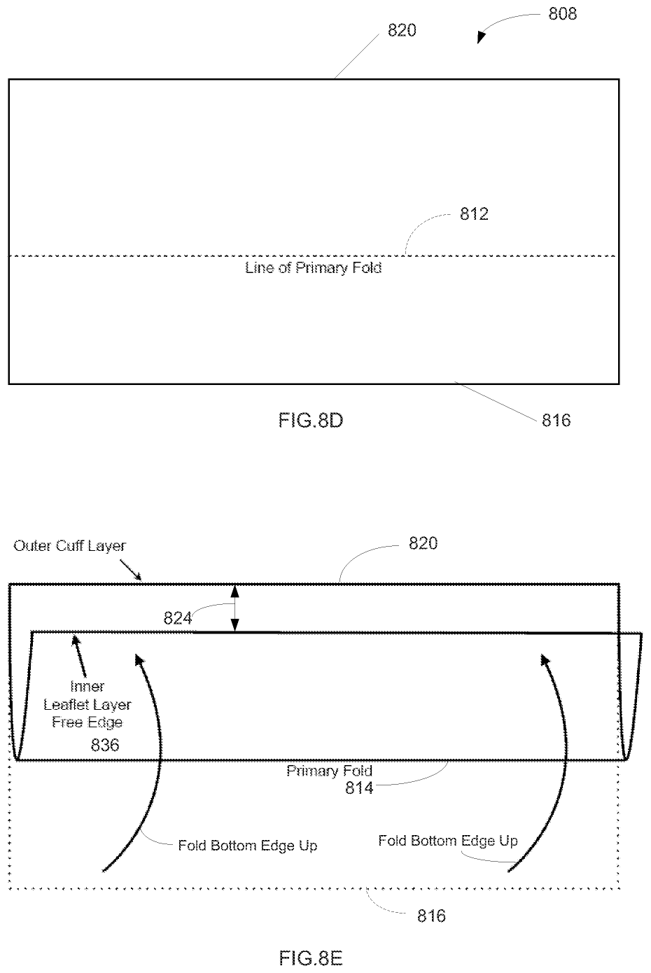

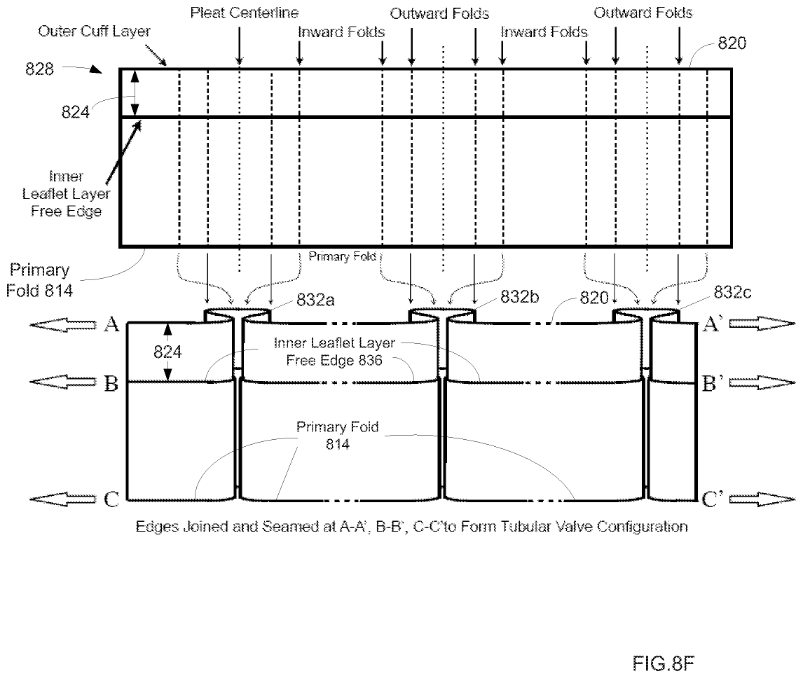

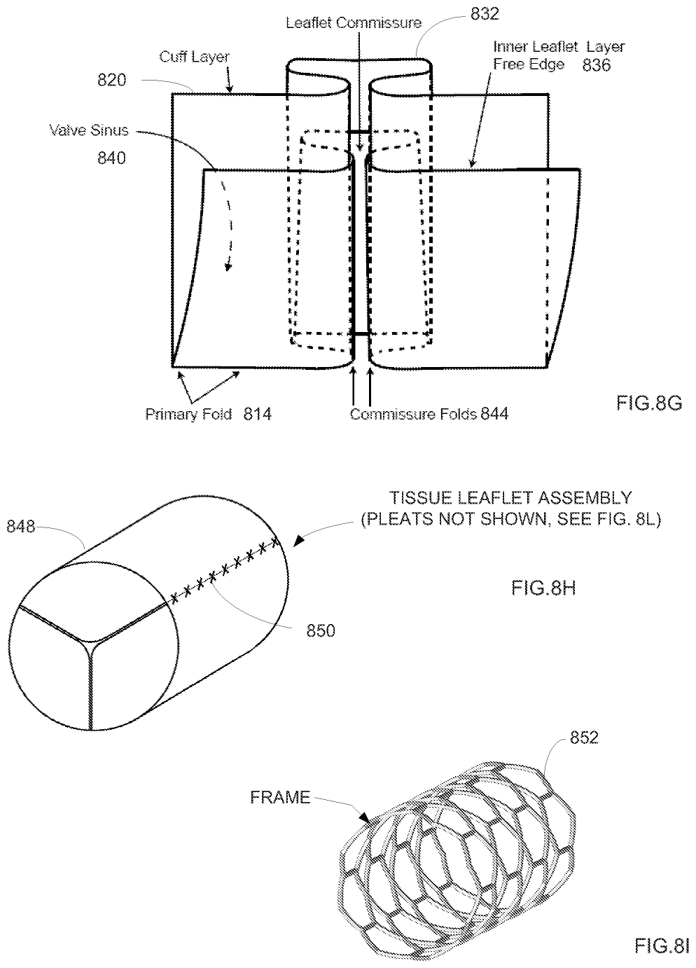

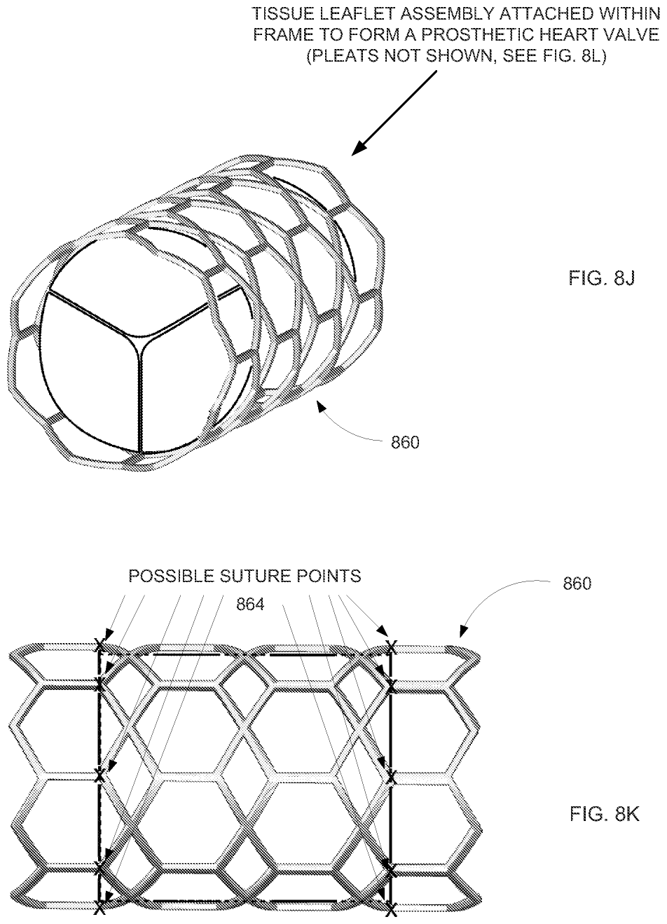

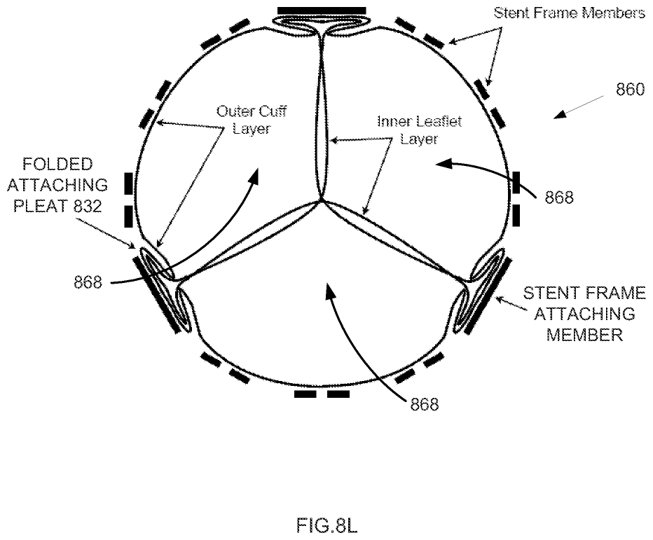

With reference now to FIG. 4, an embodiment associated with forming a tissue leaflet assembly and attachment to a frame to form a prosthetic heart valve at 400 is further described. It is to be understood that the tissue generated from one or more of the tissue preparation procedures described herein may be used for a variety of devices or uses, and that use in a prosthetic heart valve is but one possible application for utilizing the tissue. For example, the tissue may be used in a shunt, or as graft material for repair or modification of one or more human organs, including the heart and its blood vessels. By way of further example, the tissue may be used as a pericardial membrane patch for repair of congenital heart defects. The tissue also has application as a prosthetic tissue in tendon and ligament replacement, and as a tissue product for wound management. Moreover, for use in a prosthetic heart valve, the tissue may be configured in a variety of ways and attached to a frame in a variety of ways. By way of example and not limitation, in at least one embodiment, the prepared tissue is formed into a tissue leaflet assembly at 404 by folding the tissue at 408, preferably while the tissue is in a dry state, to form at least a portion of the tissue leaflet assembly. Here, those skilled in the art will appreciate that a completed tissue leaflet assembly may be formed of a single monolithic piece of tissue 800, such as that shown in FIG. 8A, or alternatively, as shown in FIGS. 8B and 8C, it may be formed of a plurality of tissue pieces 802 that are operatively connected, such as by gluing or sewing the tissue pieces together along seams 804. As seen in FIG. 8C, the seams 804 are preferably situated at overlapping portions of pleats 832 of the plurality of tissue pieces 802.

As those skilled in the art will further appreciate, a single monolithic piece of tissue 800 or a plurality of tissue pieces 802 may be used to form a prosthetic heart valve, wherein the tissue leaflet assembly is not a folded construct. By way of example and not limitation, a plurality of separate tissue pieces may each be attached to a frame (such as by suturing) to form a prosthetic heart valve. Thereafter, whether the prosthetic heart valve is made of a folded tissue leaflet assembly or a plurality of separate tissue pieces attached to a frame, the resulting prosthetic heart valve may then be further manipulated for delivery as a dry prosthetic heart valve.

In an alternative embodiment, tissue generated from one or more of the tissue preparation procedures described herein may be used to form a prosthetic heart valve that includes a frame, and that may be implanted by a “trans-apical” approach in which the prosthetic heart valve is surgically inserted through the chest wall and the apex of the heart.

In yet another alternative embodiment, tissue generated from one or more of the tissue preparation procedures described herein may be used to form a prosthetic heart valve that does not include a frame, and is not delivered via a catheter, but rather, is implanted via a surgical opening through the patient's chest. In such a case, the prosthetic heart valve may be packaged for delivery as a dry prosthetic heart valve.

In still yet another alternative embodiment, tissue generated from one or more of the tissue preparation procedures described herein may be used to form a prosthetic heart valve that includes a frame, but that is not delivered via a catheter, but rather, is implanted via a surgical opening through the patient's chest. In such a case, the prosthetic heart valve may be packaged for delivery as a dry prosthetic heart valve.

As a further alternative to the embodiments described herein, tissue may be implanted in a “wet” or hydrated state. For example, a prosthetic heart valve utilizing a prepared tissue described herein may be packaged for delivery as a hydrated prosthetic heart valve. Accordingly, while a portion of the tissue preparation process may include drying the tissue so that it may be manipulated more easily, the tissue may then be hydrated at a later point in time prior to implantation, and it may be maintained in a hydrated condition up to and including packaging, delivery and implantation into a patient. Advantages associated with using a folded tissue leaflet assembly include that a folded structure allows a relatively thin membrane to be used by avoiding suture lines in loaded, dynamically active surfaces. Accordingly, a sutureless leaflet assembly preserves long-term integrity. However, it is to be understood that a prosthetic heart valve that does not include a folded tissue leaflet assembly is encompassed by one or more embodiments described herein.

With reference now to FIGS. 8D-8L, and in accordance with at least one embodiment, for a prosthetic heart valve that includes a tissue leaflet assembly formed of a folded tissue membrane, the folding sequence for the tissue is shown for configuring the tissue into a completed tissue leaflet assembly. More particularly, a tissue blank 808 is shown in FIG. 8D, wherein the tissue blank 808 is a single monolithic piece of tissue 800. Depending upon the size requirements for a given tissue leaflet assembly, a line of primary fold or fold line 812 (shown as a dashed line) is visualized for the tissue blank 808. As shown in FIG. 8D, the primary fold 814 is achieved along the fold line 812 by folding the bottom edge 816 of the tissue blank 808 toward the top edge 820, but leaving a cuff portion 824 along the upper portion 828 of the tissue blank 808. Here, it is noted that the direction of top and bottom are relative to each other and are used as a convenience for describing the folding sequence, wherein such directions correspond to the orientation of the page illustrating the drawings. Advantageously, the folding geometry of FIGS. 8D-8L forms cuffs 824 that are continuous with the leaflets, thereby reducing the risk of aortic insufficiency or leakage.

With reference now to FIG. 8F, after folding the tissue blank 808 along fold line 812 to form primary fold 814, pleats are formed by folding the tissue along its length. For the embodiment shown in FIG. 8F, three pleats 832a, 832b, and 832c are shown. FIG. 8G illustrates a detail drawing of a single pleat 832 representative of one of pleats 832a-c. In FIG. 8G, the inner leaflet layer free edge 836 is shown, as is the valve sinus 840 and the commissure folds 844.

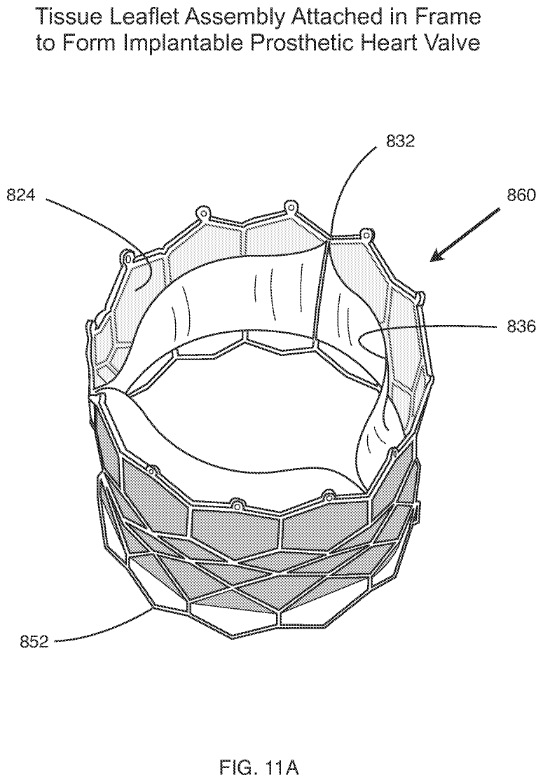

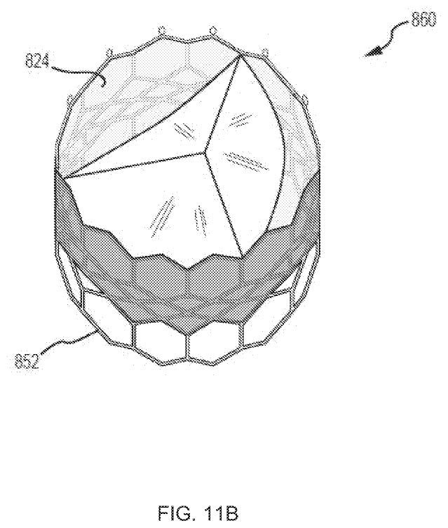

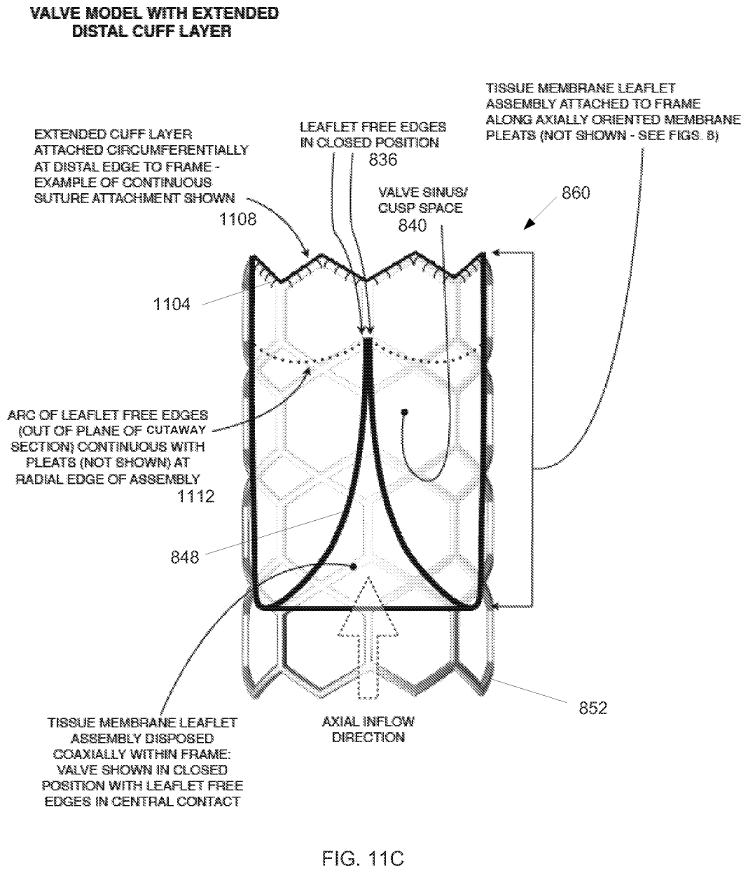

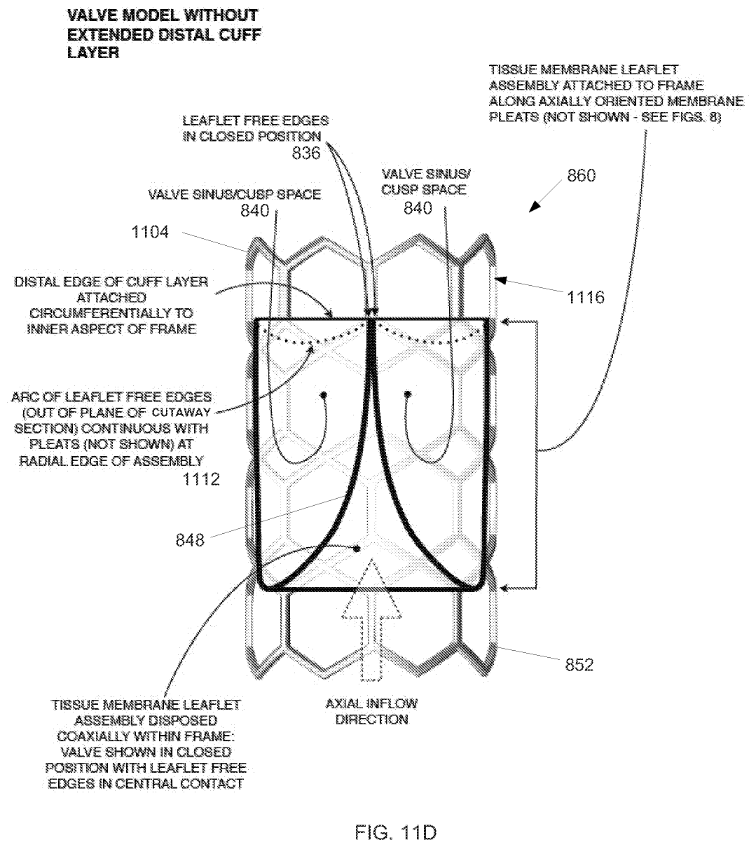

Referring again to FIG. 4 as well as FIG. 8H, at 412 the folded tissue is seamed to form a folded tissue leaflet assembly. More particularly, FIG. 8H shows a schematic perspective drawing of tissue leaflet assembly 848, wherein the pleated tissue construct shown in the bottom half of FIG. 8F is seamed, such as along seam 850, to form a substantially tubular construct. At 416, the folded tissue leaflet assembly 848 is maintained dry or is partially hydrated prior to mounting the tissue leaflet assembly in a frame. At 420, the tissue leaflet assembly 848 is then attached within a frame, such as frame 852 shown in FIG. 8I. The tissue leaflet assembly 848 attached within a frame 852 forms an implantable prosthetic heart valve 860, such as that shown in the schematic perspective drawing of FIG. 8J, side elevation view FIG. 8K, as well as that shown in the photo of FIG. 11A, and drawing of FIG. 11B. FIG. 8K illustrates possible suture points 864 where the tissue leaflet assembly 848 can be sutured to the frame 852. That is, the tissue leaflet assembly 848 may be attached within the frame 852, such as by suturing the outer layer of the tissue leaflet assembly 848 to the frame. In the foregoing sentence, and as used herein, it is noted that the term “attached” means that the tissue leaflet assembly 848 is secured to the frame 852, although the inner leaflet layer free edges 836 are able to readily move during operation of the prosthetic heart valve 860.

Referring now to FIG. 11C, a cutaway side elevation view of a prosthetic heart valve 860 that includes a frame 852 with a tissue leaflet assembly 848 attached therein is shown. The tissue membrane leaflet assembly 848 is disposed coaxially within the frame 852. As shown in FIG. 11C, the valve 860 is illustrated in the closed position with the leaflet free edges 836 in at least partial contact with each other. An arc 1112 of the leaflet free edges 836 (out of plane of the cutaway view) is continuous with pleats 832 at the radial edge of the tissue leaflet assembly 848, and may be seen in the alternate view shown in FIG. 8L. The tissue membrane leaflet assembly 848 is attached to the frame 852 along the axially oriented membrane pleats 832, as illustrated again in FIG. 8L. The extended cuff layer is attached circumferentially at the distal edge 1104 of the frame 852. By way of example and not limitation, continuous suture attachment 1108 may be used to attach the extended cuff layer to the distal edge 1104.

Referring now to FIG. 11D, an embodiment is shown wherein the cuff layer is not extended distally to the distal edge 1104 of the frame 852. As shown in FIG. 11D, the distal edge of the cuff layer is attached circumferentially to an inner aspect of the frame 852, such as along those possible suture points 864 illustrated in FIG. 8K. As a result, a distal portion 1116 of the frame 852 does not include any portion of the tissue leaflet assembly 848, such as the cuff layer. However, with the valve 860 in the closed position the leaflet free edges 836 still at least partially contact each other.

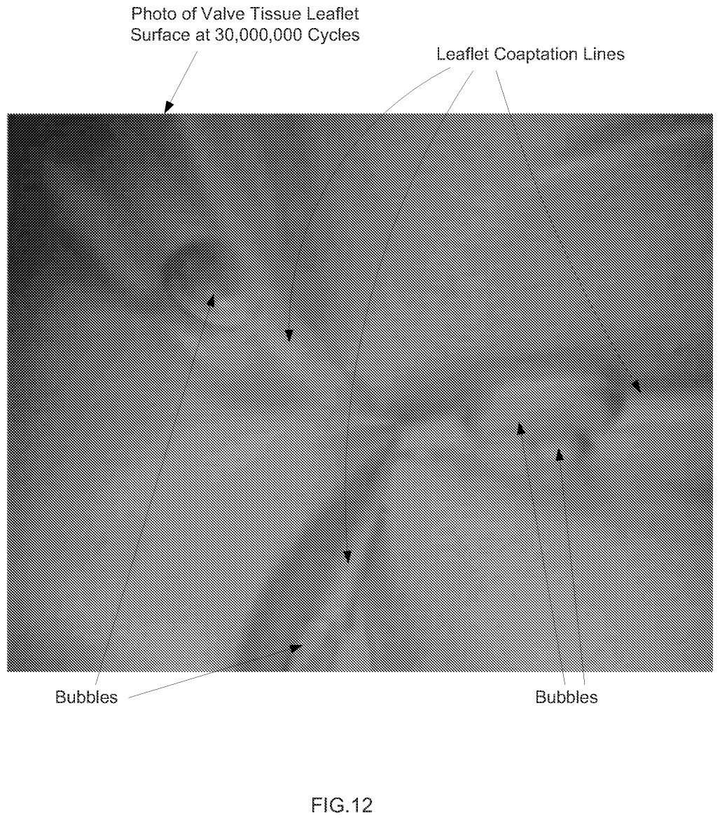

With reference now to FIG. 8L, an end view of the prosthetic heart valve is shown. As depicted in FIG. 8L, the pleats 832 are used as the portion of the tissue leaflet assembly 848 to attach to the frame 852. As can be seen in FIG. 8L, the outer cuff layer is attached to the frame members of frame 852. When the prosthetic heart valve 860 is closed, the cusps 868 formed by the inner leaflet layer are generally situated as depicted in FIG. 8L. FIG. 12 is a photo of the tissue leaflets of a prosthetic heart valve after 30,000,000 cycles of testing to model performance if associated with a human heart. In testing, the prosthetic heart valve 860 has demonstrated a natural opening gradient of approximately 5 mmHg.

It will be appreciated by one of ordinary skill in the art that the tissue leaflet assembly 848 described and shown herein is but one possible construct for forming a flow control mechanism that can be attached to a frame to regulate the flow of blood in a patient's vascular system upon deployment. That is, the illustrated tissue leaflet assembly 848 is provided by way of example and not limitation, and in no way should be interpreted to limit the geometries of membrane leaflet assemblies that can be used to regulate fluid flow. Accordingly, other leaflet configurations and constructs are considered encompassed by claims directed to or otherwise including premounted percutaneously deliverable valves.

As those skilled in the art will appreciate, the frame 852 may be a stent or a structure having similarities to a stent. The frame 852 essentially serves as a holding mechanism for the tissue leaflet assembly 848 that can then be inserted percutaneously into a patient, wherein the frame 852 serves as a way to anchor the folded tissue leaflet assembly 848 to a vascular portion (e.g., in situ arterial tissue) of the patient. Thus, at 424 the tissue leaflet assembly 848 is inserted into a frame 852. More particularly, at 424a the frame 852 may comprise a balloon-expandable frame, or alternatively, at 424b a self-expanding frame may be used. After the tissue leaflet assembly is inserted into the frame, at 428 the folded tissue leaflet assembly 848 is attached to the frame 852, such as by suturing the tissue leaflet assembly 848 to the frame 852 to form an implantable prosthetic heart valve 860, such as that shown in FIG. 8L. In at least one embodiment, after attaching the tissue leaflet assembly 848 within the frame 852 and connecting the tissue leaflet assembly 848 to the frame 852 to form an implantable prosthetic heart valve 860, at 432 the prosthetic heart valve 860 is fully hydrated for inspection and testing. Thereafter, the fully constructed implantable prosthetic heart valve 860 may be dried and maintained in a substantially dry condition. Accordingly, as those skilled in the art will appreciate, one or more embodiments described herein provide a tissue 800 suitable for implanting in a human, wherein the implantable tissue may be allowed to dry prior to implanting, or it may be hydrated prior to implanting. In addition, the tissue 800 is suitable for use in forming a tissue leaflet assembly 848 for use in a prosthetic heart valve, including an implantable prosthetic heart valve 860 that can be implanted with its tissue leaflet assembly in a dry state, or with its tissue leaflet assembly in a partially or fully hydrated state.

One or more of the embodiments of the tissue leaflet assemblies described herein may be implanted into the patient using a balloon-expandable frame or a self-expanding frame. Expandable frames are generally conveyed to the site of the target valve on balloon catheters. For insertion, the expandable frame is positioned in a compressed configuration along the delivery device, for example crimped onto the balloon of a balloon catheter that is part of the delivery device intended for coaxial mounting on a guidewire. After the expandable frame is positioned across the plane of the valve, the expandable frame is expanded by the delivery device. For a self-expanding frame, commonly a sheath is retracted, allowing expansion of the self-expanding frame.

In at least one embodiment, the frame comprises a metal alloy frame possessing a high strain design tolerance that is compressible to a relatively small diameter. By providing a device with a low profile, the implantable prosthetic heart valve 860 allows standard retrograde arterial aortic delivery via femoral artery insertion, without surgical cutdown or general anesthesia. This is achieved by providing the prosthetic heart valve on a premounted delivery system with the tissue leaflet assembly or tissue membrane construct in a substantially dry condition.

In accordance with one or more embodiments, a dry tissue membrane has substantially less mass than a wet membrane. By way of example, a substantially dry pericardium tissue prepared by one or more of the present embodiments has approximately 30% of the mass of a wet pericardium tissue, and marked reduction in profile and packing volume, thereby achieving a relatively low profile and making it suitable for implantation in greater number of patients, especially those having small diameter vascular systems. In addition, a dry prosthetic heart valve does not require storage and transport in preservative. A dry prosthetic heart valve can be mounted on a delivery catheter at its location of manufacture, which allows for pre-packaging of an integrated delivery system. In the foregoing sentence, it is noted that the term “mounted” means that the prosthetic heart valve 860 is temporarily associated with the delivery catheter. Together with a relatively low profile, embodiments of the prosthetic heart valve thereby offer reliability and convenience because the implantable prosthetic heart valve 860 is pre-mounted upon its delivery catheter and forms part of a pre-packaged delivery system. In addition, a dry prosthetic heart valve does not require rinsing, rehydration, or mounting in a catheterization lab. Therefore, a dry prosthetic heart valve can be inserted directly from package into the patient's body at a critical time during the procedure. Advantageously, this avoids procedure time, manipulation, and errors of mounting, crimping, and orienting catheters and sheaths. Once at the surgical facility/location, the dry prosthetic heart valve is inserted and delivered by balloon catheter expansion in the plane of the target valve in the standard way and the dry prosthetic heart valve begins to function immediately, even without specific steps to rehydrate the tissue membrane portion of the heart valve from its dry state, with hydration of the tissue membrane subsequently occurring rapidly and naturally in the body. More particularly, hydration of the tissue membrane portion occurs rapidly and begins with simple preparatory flushing of catheter lumens with saline. Thereafter, hydration continues with device insertion and dwelling into the central blood vessels, and completes naturally after deployment in the patient's body.

The low profile of the implantable prosthetic valve is particularly advantageous for patient's having relatively small diameter vascular systems. Table 1 provides aortic and pulmonary valve prosthesis sizing.

TABLE
Aortic and Pulmonary Valve Prosthesis Sizing

| | Collapsed Implantable | Collapsed Implantable |
|---|---|---|
| Aorta/Pulmonary Valve | Prosthetic Heart Valve | Prosthetic Heart Valve |
| Diameter | Size (French) | Diameter |
| 19-21 mm | 12 French | 4.0 mm |
| 22-26 mm | 14 French | 4.7 mm |
| 27-30 mm | 16 French | 5.3 mm |

For most human patients, the femoral artery has a diameter of between about 5-8 mm. Accordingly, it is apparent that embodiments of the collapsed implantable prosthetic heart valves 860 described herein offer a low profile that enables a larger group of patients to qualify for receiving an implantable prosthetic heart valve 860. As a result of the sizing advantages offered by one or more embodiments of implantable prosthetic heart valves 860 described herein, virtually no candidate patients would be excluded from treatment with an implantable prosthetic heart valve 860 without open heart surgery and without general anesthesia on the basis of inadequate femoral blood vessel access caliber. In addition, one or more embodiments of the implantable prosthetic heart valve 860 described herein feature a scalable construct, wherein the implantable prosthetic heart valves 860 can be produced to accommodate target valve diameters ranging between 6-35 mm, and wherein the implantable prosthetic heart valves 860 offer consistent function using fundamentally a single design.

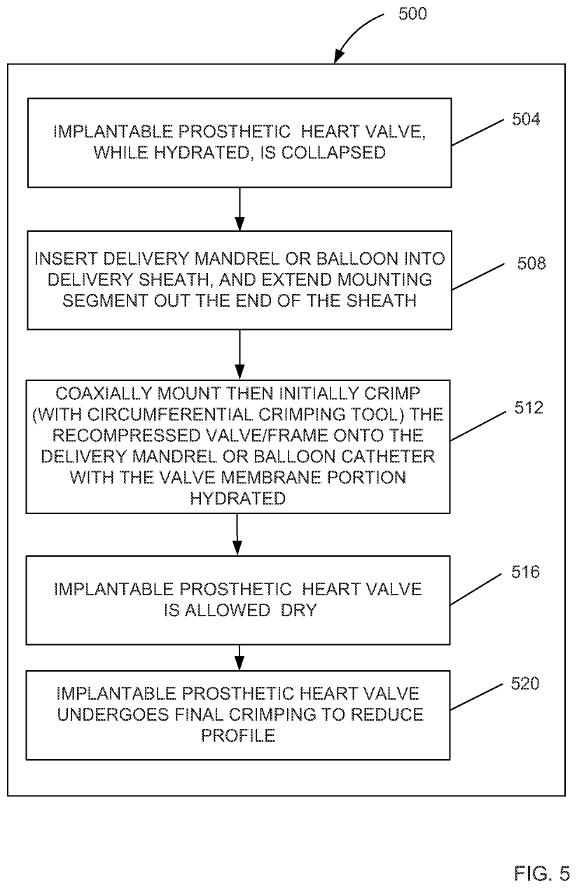

Referring now to FIG. 5, the mounting of the implantable prosthetic heart valve 860 into a delivery system at 500 is further described. More particularly, at 504 an implantable prosthetic heart valve 860 (also referred to herein as a percutaneously deliverable heart valve) is collapsed. The initial phase of collapsing the percutaneously deliverable heart valve is executed with the tissue membrane in a hydrated condition. That is, since the percutaneously deliverable heart valve 860 includes the frame 852 with the tissue leaflet assembly 848 attached within the frame 852, the percutaneously deliverable heart valve 860 is collapsed down as an integral unit. If a balloon-expandable frame is used, then an axial puller may be utilized to collapse down the frame 852 of the percutaneously deliverable heart valve 860 without the application of force directly to the sides of the frame 852. This procedure offers the advantage of preserving the cell structure of the frame 852 while also maintaining the orientation of the leaflets of the tissue leaflet assembly 848 as the percutaneously deliverable heart valve 860 is compressed. The proper orientation and disposition of the leaflets is facilitated by the hydrated state of the leaflets. This assists in preventing tissue prolapse or bulging of the tissue 800 or 802 through the frame 852. In addition, this technique reduces recompression strain on the metal frame 852 (e.g., a stent) that can tend to compromise fatigue life of the frame 852. This technique also tends to promote the circumferentially uniform collapsing of cells in the frame 852, thereby mitigating bunching of the tissue that forms the tissue leaflet assembly 848 of the percutaneously deliverable heart valve 860. For a self-expanding frame, the sides are forced to collapse by providing a radial compression force to the frame and may be assisted by axial traction force.

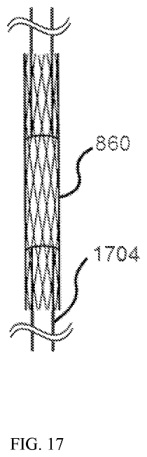

With further reference to FIG. 5, the percutaneously deliverable heart valve 860 (i.e., the frame 852 with the tissue leaflet assembly 848 attached thereto) is collapsed in an initially hydrated state. At 508 the delivery mandrel 1704 (as best seen in FIG. 17) or balloon is inserted into a delivery sheath, and the mounting segment is then extended out the end of the sheath. Thereafter, at 512 the sheath and frame are coaxially mounted and then compressed with initial crimping onto the mounting segment with the tissue leaflet assembly 848 still in a hydrated state. At 516, the tissue leaflet assembly 848 of the percutaneously deliverable heart valve 860 is then allowed to dry, which further reduces the volume and profile of the tissue membrane leaflets, permitting further compression by radial force. Accordingly, in the final compression step, the percutaneously deliverable heart valve 860 is then further crimped with a circumferential crimping tool at 520 to finally mount the compressed valve/frame onto the delivery mandrel or balloon catheter.

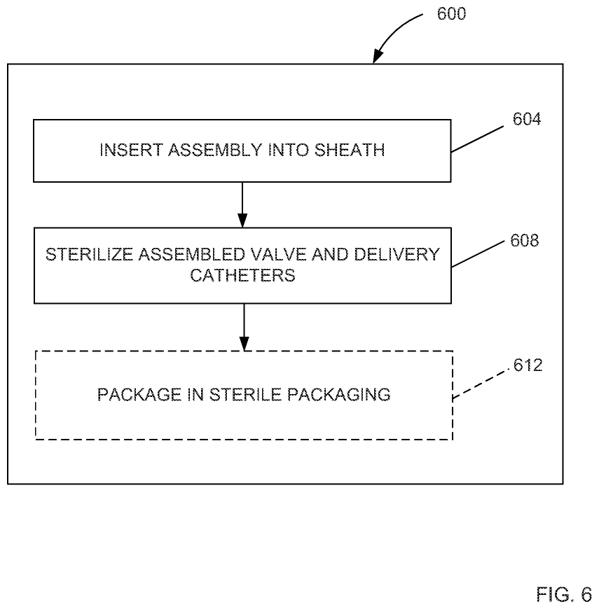

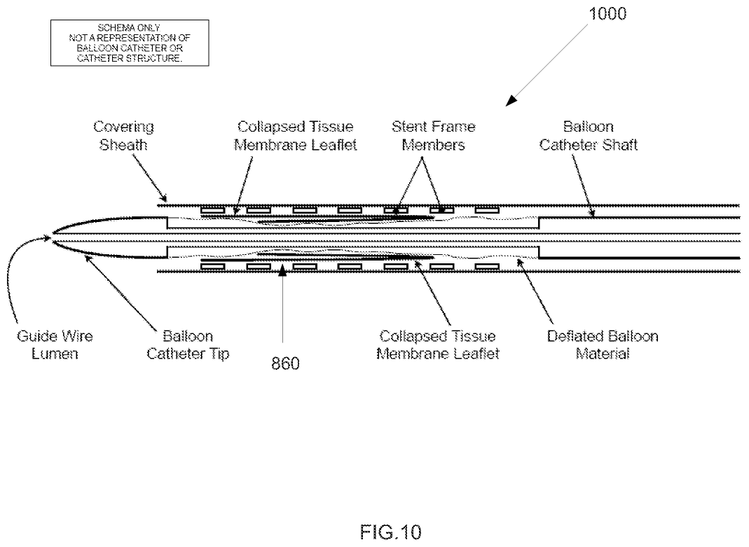

Referring now to FIG. 6, the ensheathing, sterilization and packaging at 600 is described. More particularly, once the percutaneously deliverable heart valve 860 is coaxially mounted and crimped on a delivery mandrel or balloon catheter as described above and shown in FIG. 5, the assembly is then inserted at 604 into a distal end of a delivery sheath, such as by “backloading” the assembly into position with a distal end of the percutaneously deliverable heart valve 860 contained within the delivery sheath proximate the end of the sheath. Reference here is made to FIG. 10 that schematically illustrates catheter 1000 with an implantable prosthetic heart valve 860 mounted thereto.

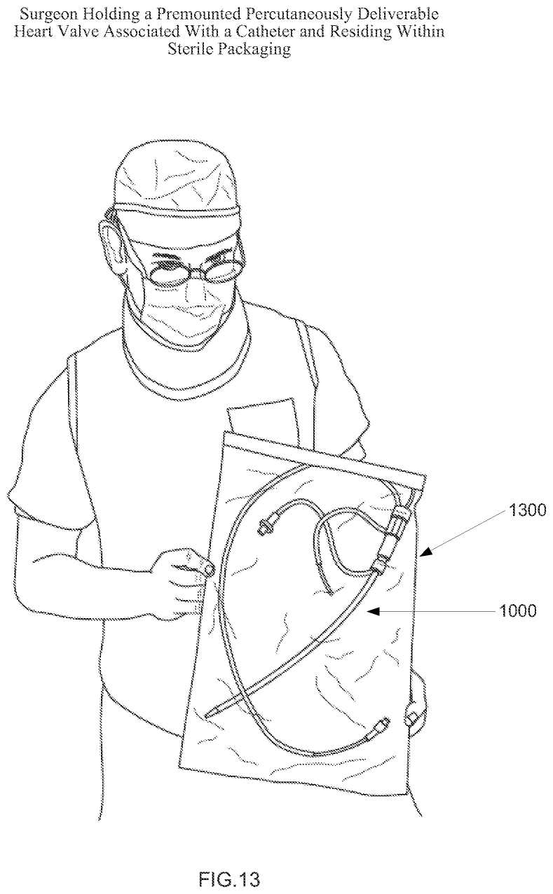

With further reference to FIG. 6, at 608 the percutaneously deliverable heart valve 860 and delivery catheters are sterilized, such as by using by one or more of ethylene oxide, proton beam, or gamma radiation. At 612, the assembly is then optionally packaged in a sterile package. Additional elements are optionally shipped with the assembly, wherein, by way of example, such elements may include any necessary delivery tools and documentation. In at least one embodiment, the package may optionally contain a device to control the water vapor content within the sealed volume of the package. FIG. 13 depicts a surgeon holding a sterile package 1300 containing a premounted percutaneously implantable prosthetic heart valve.

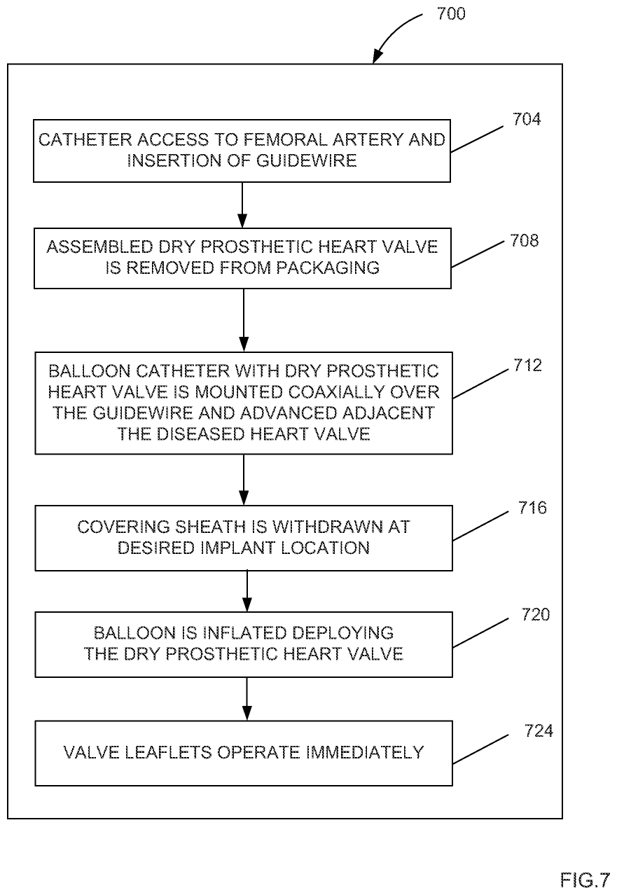

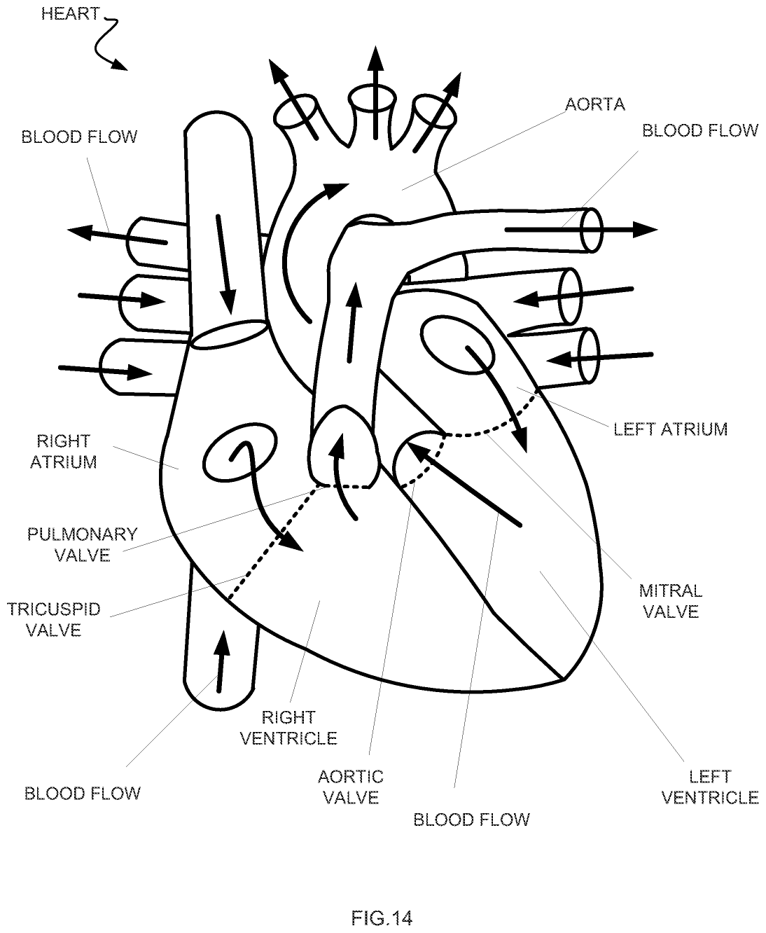

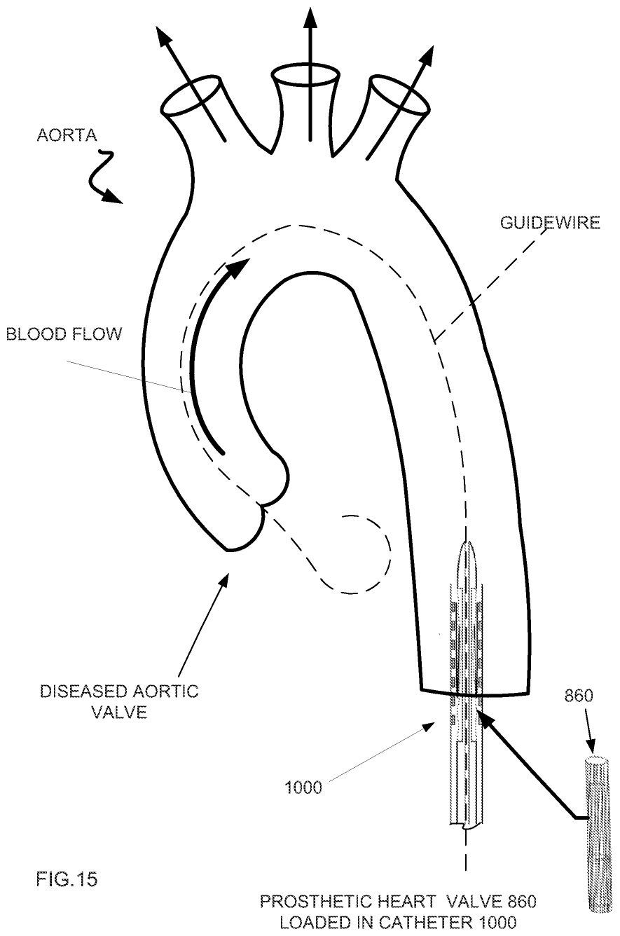

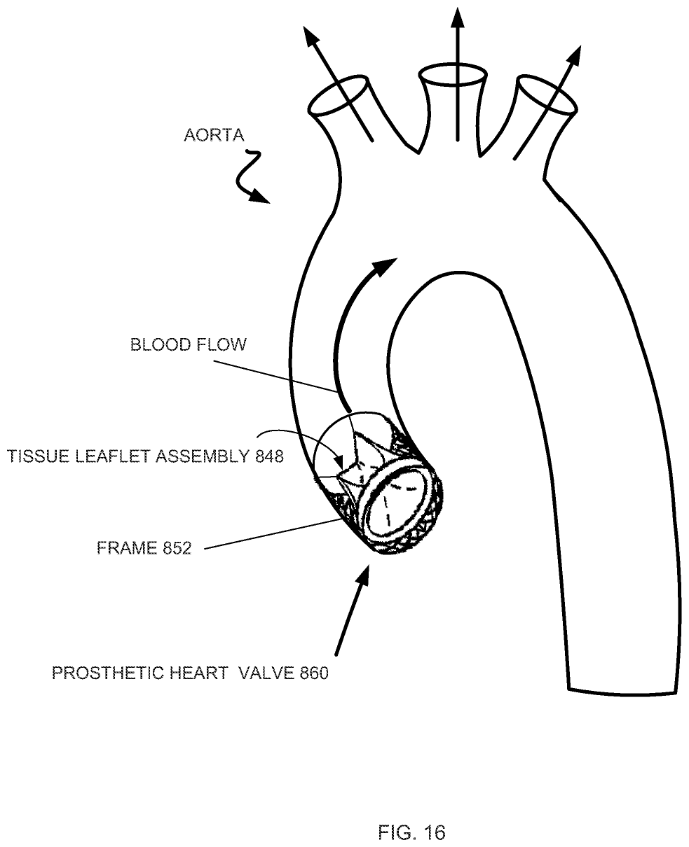

Referring now to FIG. 7, a flow chart illustrating the general procedure associated with implantation of the percutaneously deliverable heart valve 860 is provided. More particularly, at 704, catheter access is gained to the patient's femoral artery and a guidewire is placed through the plane of the diseased valve that is targeted to receive the implant. FIG. 14 is a schematic of a simplified cutaway view of a human heart, including heart valves that may be targeted for receiving an embodiment of an implantable prosthetic heart valve. FIG. 15 illustrates the aorta with the guidewire placed through the diseased aortic valve. At 708, the percutaneously deliverable heart valve 860 in the form of a prepackaged assembled dry prosthetic heart valve is removed from the sterile packaging. The dry prosthetic heart valve assembly, including its lumens, are preferably flushed and prepared in the usual fashion for standard balloons and catheters that do not contain a biocompatible tissue. Advantageously, implantation of the dry prosthetic heart valve assembly can be conducted without specific maneuvers for rehydration of the tissue leaflet assembly 848 of the percutaneously deliverable heart valve 860. Some rehydration of the tissue leaflets may occur as a consequence of the routine flushing of the catheter lumens in preparation for use as with any other catheters. Additionally, implantation of the dry prosthetic heart valve assembly can proceed without additional cleaning steps, such as by having to use alcohol or water rinsing solutions. In addition, further mounting of the dry tissue leaflet assembly 848 that resides in the frame 852 of the percutaneously deliverable heart valve 860 is not needed, thereby obviating the need for another mounting step. Accordingly, the percutaneously deliverable heart valve 860 can essentially be implanted percutaneously in its dry state. At 712, the carrier catheter or balloon catheter is then coaxially mounted and advanced over the guidewire, such as under fluoroscopic vision initially to the level of the great vessel where it can be inspected under fluoroscopy. At 716, and after the nominal position and configuration is confirmed, the delivery system is advanced through the plane of the diseased valve under fluoroscopy, and the covering sheath is withdrawn, either at this point or during the advance prior to it, thus exposing the mounted implantable prosthetic heart valve 860 in place. At 720, in the case of a balloon expandable frame, and assuming the delivery approach involving the pre-mounting of the percutaneously deliverable heart valve 860 on the expansion balloon, the balloon is then inflated, deploying the percutaneously deliverable heart valve 860 in the plane of the valve. At 724, the leaflets of the percutaneously deliverable heart valve 860 operate immediately. The deployed prosthetic heart valve 860 is shown in FIG. 16, wherein the tissue leaflet assembly 848 serves to properly control the flow blood.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

The one or more present inventions, in various embodiments, include components, methods, processes, systems and/or apparatus substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes (e.g., for improving performance, achieving ease and/or reducing cost of implementation).

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention (e.g., as may be within the skill and knowledge of those in the art, after understanding the present disclosure). It is intended to obtain rights which include alternative embodiments to the extent permitted, including alternate, interchangeable and/or equivalent structures, functions, ranges or acts to those claimed, whether or not such alternate, interchangeable and/or equivalent structures, functions, ranges or acts are disclosed herein, and without intending to publicly dedicate any patentable subject matter.

1.-6. (canceled)

7. An assembly configured for replacing an aortic or pulmonary heart valve in a human patient, comprising:
a transcatheter, prosthetic aortic or pulmonary heart valve, including:
a frame that is expandable and collapsible; and
a tissue leaflet assembly sutured directly to the frame, wherein the tissue leaflet assembly includes three substantially dry leaflets that each include a free edge that has a curved profile configured to increase total surface area between coapting leaflets, wherein a single piece of treated mammalian pericardial tissue is used to form the three substantially dry leaflets, the mammalian pericardial tissue being treated with glutaraldehyde and glycerol and having a thickness of between about 50 to 300 μm,
wherein the frame and the tissue leaflet assembly are collapsible as an integral unit such that a cell structure of the frame and an orientation of the leaflets of the tissue leaflet assembly are maintained during a collapsing of the transcatheter, prosthetic aortic or pulmonary heart valve;
a percutaneously insertable valve delivery mechanism, wherein the transcatheter, prosthetic aortic or pulmonary heart valve is releasably and coaxially mounted onto a balloon catheter of the percutaneously insertable valve delivery mechanism, wherein the balloon catheter has a size of 12 to 14 French, and wherein the balloon catheter includes a guidewire lumen; and
a sterile package containing the transcatheter, prosthetic aortic or pulmonary heart valve that is releasably and coaxially mounted onto the balloon catheter.

8. The assembly of claim 7, wherein the frame comprises a stent.

9. The assembly of claim 7, wherein the treated mammalian pericardial tissue has an ultimate tensile strength of greater than about 12 MegaPascals.

10. The assembly of claim 7, wherein the treated mammalian pericardial tissue does not include a matrix that has been exposed to a polymer infiltrate.

11. The assembly of claim 7, wherein the treated mammalian pericardial tissue has been treated with distilled water.

12. The assembly of claim 7, wherein the treated mammalian pericardial tissue has been treated with isopropyl alcohol.

13. The assembly of claim 7, wherein the tissue leaflet assembly comprises two or more integrated cusp and leaflet folded structures that each include a mobile leaflet section and a cuff wall section located radially outside the mobile leaflet section such that in a closed position at least a portion of free edges of the mobile leaflet sections are in contact with each other.

14. The assembly of claim 7, wherein the tissue leaflet assembly comprises one or more pleats that are sutured to the frame.

15. The assembly of claim 7, wherein the cuff section extends longitudinally beyond the free edges of the mobile leaflet sections.
